(19) United States
(12) Patent Application Publication
(10) Pub. No.: US 2016/0223007 A1
(43) Pub. Date: Aug. 4, 2016

(54) SCREW WITH AN ELLIPTICAL LONGITUDINAL AND/OR CROSS SECTION

(71) Applicant: CeramTec GmbH, Plochingen (DE)

(72) Inventors: Heinrich Wecker, Eckental (DE); Alfons Kelnberger, Rothenbach (DE); Mateusz Juszczyk, Velden (DE)

(21) Appl. No.: 14/916,403

(22) PCT Filed: Sep. 5, 2014
(86) PCT No.: PCT/EP2014/068902

§ 371 Date: Mar. 3, 2016

(30) Foreign Application Priority Data
Sep. 5, 2013 (DE) 10 2013 217 734.8

(51) Int. Cl. F16B 25/00; A61C 8/02; A61C 8/00; A61B 17/70; F16B 33/00; F16B 39/284

(52) U.S. Cl. CPC F16B 25/0078; F16B 33/006; F16B 25/0015; F16B 39/284; A61C 8/0022; A61B 17/7001; A61C 8/0013; A61C 8/0006; A61B 2503/40

(57) ABSTRACT

A screw which is self-locking screw has an elliptical screw body. The screw may be used, e.g., to secure implants such as spinal implants or, e.g., to secure bone or plants.

The invention relates to a screw. In particular, the invention relates to a self-locking screw.

A screw is a pin or bolt which is provided externally with a thread and has a screw head with a screw head drive. A connection produced by a screw is positively engaged and can usually be released again.

There are numerous different geometric variations with respect to thread shapes and the screw head or screw head drives. Likewise, a number of techniques are known for securing screws, in particular for securing them against release. These are generally based on the screw head being secured against subsequent turning. Examples of such screw securing means are splints, lock nuts, the most varied versions of washers and many others.

A further variant of the screw securing means consists of jamming or adhesion of the thread of the screw with the material into which the screw is introduced. This can be achieved by an additionally introduced bonding adhesive or by a coating of the screw thread.

However, these methods for securing the screw against subsequent loosening have the disadvantage that they require materials which have a relatively high modulus of elasticity, i.e. materials which are rigid are required. On the other hand, the screws should have a lower modulus of elasticity, that is to say they should be rather elastic in order to achieve a durable connection. Another disadvantage is that the screw per se is not sufficient in order to achieve securing, but other components or materials are required in order to secure the screw.

Moreover, however, the connections can be problematic in the case of materials which react elastically or deformably. The screw holes quickly become deflected and thus a reduced or insufficient friction occurs between the screw and the material. As a result a screw can be easily loosened.

However, if a reliable screw connection is absolutely necessary, such loosening cannot be tolerated. An example of this is an implant which is intended to be firmly connected to a bone by means of a screw connection. Failure of the screw connection may have fatal consequences, since in this case not only is a further operation necessary, but also secondary injuries to the surrounding tissue can be caused by the loosened implant.

The object of the invention is to provide a screw which without the aid of other components produces a permanently secure connection to a material, in particular to an elastic material. The connection should preferably be positively engaged and should not become loose by itself. However, the connection produced can preferably be released again intentionally.

This object is achieved by a screw with the features according to claim 1. The screw according to the invention has a screw body with a thread, wherein the screw body is elliptical. According to particularly preferred embodiments of the invention the screw body has an elliptical cross-section and/or an elliptical longitudinal section.

In a screw body with an elliptical cross-section the ratio of the two elliptical half-axes R/r and thus the shape of the elliptical cross-section is substantially dependent upon the elasticity of the medium into which the screw is screwed. In this case “R” designates the length of the large half-axis and “r” designates the length of the small half-axis of the ellipse.

In the case of an extremely rigid material, that is to say a material with a high modulus of elasticity, the ratio R/r should tend towards 1, since otherwise the screw cannot be screwed into the medium.

In the case of an extremely elastic material, that is to say a material with a low modulus of elasticity, which allows an elastic deformation up to 100%, the ratio R/r can rise up 2 without plastically deforming or destroying the medium. Therefore, the ratio R/r is preferably between 1.01 and 2.0.

If a human bone with an elastic deformation of the order of magnitude of 1% is assumed, the ratio R/r can be selected at up to 1.01 without damage to the bone material.

A screw which is elliptically dimensioned in cross-section in such a way has, by comparison with the screws which are otherwise dimensioned with a round cross-section, a significantly higher intrinsic protection against release, which results substantially from the interaction of the elasticity of the medium or material into which the screw according to the invention is screwed, and the ellipticity, that is to say the ratio of the two half-axes R/r.

In the case of a biologically vital material such as bone as medium, there is a further feature to be considered:

Under certain biological-mechanical conditions, damaged bone material can regenerate and/or regrow, i.e. the ratio of R/r can also be chosen to be greater than the elasticity of the bone would allow in principle.

For example, a recess can be pre-drilled which with regard to the radius is oriented on the greater half-axis R. A certain primary stability results from the fact that in the region of the main axis the thread forms a mechanical connection to the material, in particular in this case the bone. Due to the bone growth or the osseointegration of the screw, in the further course of time the primary stability is substantially improved, so that a substantially higher secondary stability is achieved. The self-locking of the screw according to the invention is achieved by the screw growing into the bone. Because of its elliptical screw body the screw cannot be loosened or unscrewed by itself.

In a second variant and making use of the elasticity of the medium into which the screw is screwed, or the regenerative capacity of a vital medium, not only the cross-section of the screw body but also or only the longitudinal profile of the screw body can be elliptical. Thus, the screw body can have an elliptical longitudinal section. With respect to the dimensioning of this second variant, similar considerations to those in the first variant apply in principle: the screw body can deviate more markedly from a cylindrical screw body the more elastic the material is into which it is to be screwed.

For definition of such a screw body the ratio of the greatest diameter or of the diameter at the broadest point on the screw body D to smallest diameter or to the diameter at the narrowest point on the screw body d can be determined, that is to say D/d. This relationship is greater the more markedly the longitudinal section of the screw body deviates from the usual cylindrical shape. Since the cylindrical shape has a ratio 1/1=1, the ratio in the case of an elliptical longitudinal section must be greater than 1.

For determination of the diameter of the narrowest point on the screw body, only the screw body per se is considered, that is to say the part which usually has a thread. The lowermost part of a screw, which is frequently tapered in order to simplify the screwing, is not taken into consideration in this case. However, it may also be pointed out that the part of the screw body to be considered does not actually have to have a continuous thread.

Both variants are independent of one another, but can also be combined with one another or with all conventional variants of the prior art.

For example, an elliptical cross-section with a cylindrical longitudinal profile or a round cross-section with an elliptical longitudinal profile is conceivable.

The screw according to the invention is suitable in particular for media or materials which have a certain elasticity and/or are vital, for example biologically active, i.e. which can live, regrow, regenerate or heal. Thus the screws are suitable for use in humans, animals and plants, in particular trees, shrubs or even bamboo.

The screws are used particularly preferably as bone screws, for example in order to secure implants. Without limiting the generality, the screws are suitable for securing spinal implants for fusion of vertebral bodies or for replacement of discs, for securing trauma products of any type or also dental implants.

With respect to the aforementioned elastic materials, polymer-based materials are particularly suitable, for example.

These examples are not intended to be limiting, but merely serve for explanation of the fundamental principle of the invention.

The invention is limited to specific thread shapes. It is possible to use all conventional shapes known in the prior art which take into account the requirements of the selected screw material. In particular, all thread shapes which can be used in cancellous bone screws or cortical bone screws are also suitable.

The same applies to the screw head and screw drive, wherein elliptically shaped screw heads, which when countersunk into the medium additionally contribute to the self-locking according to the invention, are particularly suitable according to the invention. A further preferred embodiment of a screw head has on its underside, that is to say the side which is mechanically connected to the material, teeth or barbs which facilitate screwing-in without problems, but offer resistance to unscrewing of the screw by catching in the material.

All known materials which are used according to the prior art, in particular from the class of metallic materials, plastics, but also ceramic and ceramic-based materials, are suitable as screw material.

The last-mentioned group of materials includes in particular all aluminum oxide-based and zirconium oxide-based materials as well as composite materials based on these materials, but also non-oxidic ceramic materials, such as for example silicon nitride-based or silicon carbide-based materials as well as composite materials based on these materials.

Within the context of the invention it is advantageous to increase the surface of the screw body by suitable techniques and/or to modify this surface by suitable coatings so that the highest possible osseointegration rate is set. For example, at least a part of a surface of the screw body can be roughened.

In this respect, all methods known from the prior art, including mechanical and/or chemically-based methods, are suitable in principle for surface treatment. Mechanical methods for changing the roughness of the surface comprise for example methods such as grinding, polishing, blasting, chemical etching and plasma coating and/or plasma activation.

Methods of spraying additional ceramic layers to produce a larger surface are particularly suitable for ceramic screw materials. For example, a ceramic slip can be applied which either already contains pore-forming agents, or in which after application of the slip the pore-forming agents are introduced into the surface thereof and then are burned out, preferably without residue. Naturally, all other ceramic methods which produce an open porous ceramic matrix function in the same way.

All types of coatings which promote osseointegration, for example based on hydroxylapatite or tricalciumphosphate or similar bioceramic compositions, all types of bioglass coatings and all types of molecules which guarantee an effective docking of bone cells onto the screw surface securely and permanently, are suitable for the screws according to the invention for functionalization of the surfaces at least of the screw body for the purpose of increasing the osseointegration capability or the osseointegration rate. Corresponding substances are known to the person skilled in the art from the prior art.

Also, metallic coating such as thin titanium plasma coatings or titanium coatings physically deposited in another way can support the concept according to the invention.

The invention is explained in greater detail below with reference to the drawings. In the drawings:

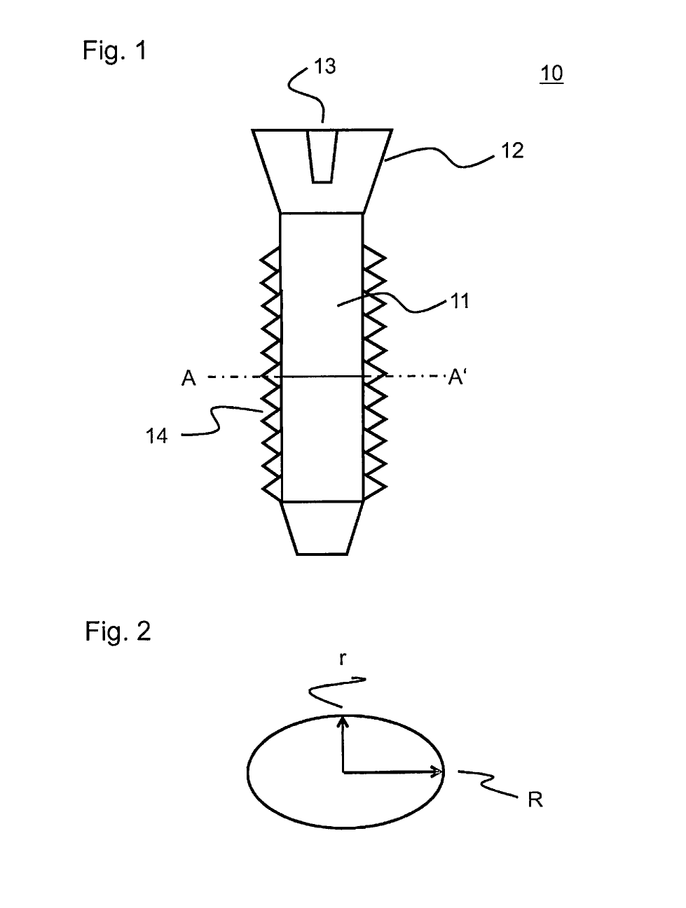

FIG. 1: shows the screw according to the invention in a longitudinal section

FIG. 2: shows a section through a screw body with an elliptical cross-section

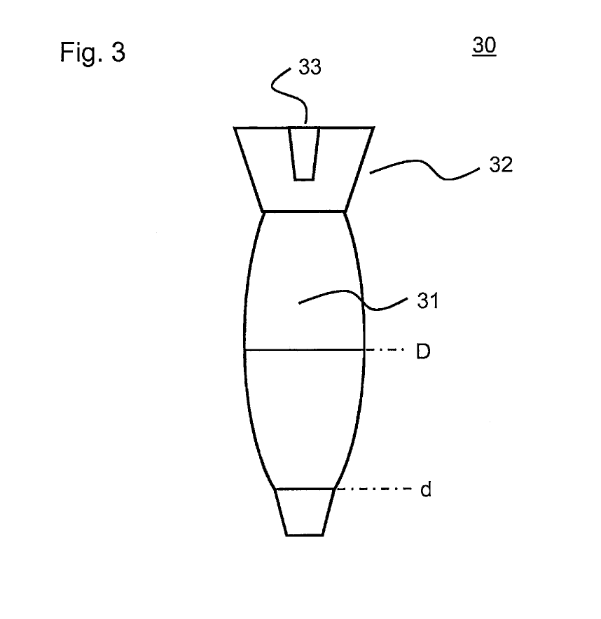

FIG. 3: shows a section through a screw body with an elliptical longitudinal section

FIG. 1 shows a screw 10 according to the invention in a longitudinal section. The screw 10 has a screw body 11 with a thread 14 and a screw head 12 with a head drive 13. The screw body 11 is cylindrical in longitudinal section.

According to a particularly preferred embodiment of the invention, the screw body 11 is shaped elliptically in cross-section. For clarification, a corresponding cross-section is illustrated in FIG. 2, wherein the line A-A′ shows the sectional plane in FIG. 1.

The elliptical cross-section of the screw body 11 has a main axis and a minor axis, wherein the lengths of the respective half-axes R and r define the shape of the elliptical cross-section. The ratio between R and r, R/r, is preferably between 1.01 and 2.0.

FIG. 3 shows a further embodiment of the invention. As in the example according to FIG. 1, the screw 30 has a screw body 31 and a screw head 32 with a head drive 33. Naturally, the screw 30 also has a thread at least on parts of the screw body 31, but this thread is not illustrated in the drawing.

In contrast to the example illustrated in FIG. 1, the screw 30 does not have a screw body which is cylindrical in longitudinal section, but has a screw body 31 which is elliptical in longitudinal section.

The cross-section of the screw body can be round, as is usual in the case of screws. However, according to a particularly preferred embodiment of the invention, the screw body can also be elliptical in cross-section. Thus, in this case both the cross-section and also the longitudinal section through the screw body would be elliptical.

Preferably the ratio of D/d, which is formed from the ratio of the diameter at the broadest and the narrowest point or also from the greatest and the smallest diameter of the screw body, is greater than 1.

1.-14. (canceled)

15. A screw comprising:
a screw body having a thread,
wherein the screw body is elliptical.

16. A crew according to claim 15, wherein the screw body has an elliptical cross-section.

17. A screw according to claim 16, wherein a ratio of a length R of a large half-axis to a length r of a small half-axis of the elliptical cross-section such that R/r is between 0.01 and 2.0.

18. A screw according to claim 15, wherein the screw body has an elliptical longitudinal section.

19. A screw according to claim 18, wherein the ratio of the greatest diameter of the longitudinal section to the smallest diameter of the longitudinal section d is greater than 1.

20. A screw according to claim 15, wherein the screw body has an elliptical cross-section and an elliptical longitudinal section.

21. A screw according to claim 15, wherein at least the screw body is made from a metallic or a ceramic material, a ceramic composite material or a plastic.

22. A screw according to claim 15, wherein at least a part of the screw body is provided with a coating.

23. A screw according to claim 22, wherein the coating promotes the osseointegration.

24. A screw according to claim 15, wherein at least a part of a surface of the screw body is roughened.

25. A screw according to claim 15 in a biological material.

26. A screw according to claim 25, wherein the biological material is a living, regenerating or healing material.

27. A screw according to claim 15, wherein the screw is in a bone.

28. A screw according to claim 27, wherein the bone is a human or animal bone.

29. A screw according to claim 15 in an implant or transplant.

30. A screw according to claim 29, wherein the implant is a spinal implant or a dental implant.

31. A screw according to claim 15 in a trauma product.

32. A screw according to claim 25, wherein the biological material is a plant.

33. A screw according to claim 32, wherein the plant is selected from the group consisting of a tree, a shrub and bamboo.

34. A screw according to claim 15 in an elastic material.

35. A screw according to claim 34, wherein the elastic material is a polymer-based material.
